(19) United States
(12) Patent Application Publication
(10) Pub. No.: US 2008/0076968 A1
(43) Pub. Date: Mar. 27, 2008

(54) Surgical tool

(75) Inventors: Andrew S. Bollier, Fort Wayne, IN (US); Vincent P. Tippmann SR., Fort Wayne, IN (US)

Correspondence Address: BIRCH STEWART KOLASCH & BIRCH, PO BOX 747, FALLS CHURCH, VA, 22040-0747, US

(73) Assignee: ZEMCO MANUFACTURING INC., Fort Wayne, IN (US)

(21) Appl. No.: 11/798,879

(22) Filed: May 17, 2007

Related U.S. Application Data
(60) Provisional application No. 60/801,415, filed on May 19, 2006.

(51) Int. Cl. A61B 1/32

(52) U.S. Cl. 600/227

(57) ABSTRACT

A device for maintaining an incision in an opened condition during an operation. A holder is provided having a distal end and a proximal end. At least one projection is provided at the distal end of the holder. The at least one projection is formed to engage a peripheral edge of an incision for maintaining the incision in an open condition. A predetermined weight is formed on the proximal end of the holder for providing a predetermined gravitational force for maintaining the holder in a predetermined position for maintaining the incision in an open condition.

### CROSS-REFERENCE TO RELATED APPLICATIONS

The present application claims priority under 35 USC 119(e) to Provisional Patent Application No. 60/801,415 filed on May 19, 2006 the entire contents of which are hereby incorporated by reference.

### BACKGROUND OF THE INVENTION

1. Field of the Invention

A surgical instrument for maintaining an incision in an opened condition during an operation includes a holder having a distal end and a proximal end. At least one projection is provided at the distal end of the holder that is adapted to engage a peripheral edge of an incision for maintaining the incision in an open condition. A predetermined weight is formed on the proximal end of the holder for providing a predetermined gravitational force for maintaining the holder in a predetermined position for maintaining the incision in an open condition.

2. Description of Background Art

Heretofore when operating on a patient wherein an incision is needed to gain access to an internal part of the patient such as a knee or stomach operation, the operation requires a doctor to perform the surgery and another doctor or nurse to hold the incision open during the surgery.

Such an operation results in an increase in cost due to the fact that a second doctor or a nurse is needed to maintain the incision in an open condition. In addition, the hands of the second doctor or nurse are right in the area of the surgeon during the surgery and may be subject to an accidental needle puncture, a cut or other mishap.

### SUMMARY AND OBJECTS OF THE INVENTION

It is an object of an embodiment of the present invention to provide a surgical instrument for maintaining an incision in an open condition wherein only a single surgeon is required to perform the operation. Thus, the cost of performing the operation is greatly reduced.

Another object of an embodiment of the present invention is to provide a surgical instrument wherein an incision may be maintained in an open condition with the use of a holder positioned at one or a plurality of locations along a peripheral edge of one or both sides of the incision.

These and other objects of the present invention are accomplished in an embodiment of the present invention by providing a surgical instrument for maintaining an incision in an opened condition during an operation that includes a holder having a distal end and a proximal end. At least one projection is provided at the distal end of the holder for engaging a peripheral edge of an incision for maintaining the incision in an open condition. A predetermined weight is formed on the proximal end of the holder for providing a predetermined gravitational force for maintaining the holder in a predetermined position for maintaining the incision in an open condition.

Further scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The present invention will become more fully understood from the detailed description given hereinbelow and the accompanying drawings which are given by way of illustration only, and thus are not limitative of the present invention, and wherein:

FIG. 1 is a perspective view of a surgical tool according to a first embodiment of the present invention;

FIG. 2 is a side view of the surgical tool illustrated in FIG. 1;

FIG. 3 is a perspective view of a surgical tool according to a second embodiment of the present invention;

FIG. 4 is a side view of surgical tool illustrated in FIG. 3;

FIG. 5 is a perspective view of a surgical tool according to the second embodiment illustrating a plurality of various projecting members and weights that may be attached relative to each other depending on conditions of a particular surgery;

FIG. 6 is a perspective view of a surgical tool according to the second embodiment of the present invention as illustrated in FIG. 3 with the weight disconnected from the projecting member and a plurality of additional weights being illustrated for attachment thereto depending on conditions of a particular surgery;

FIG. 7 is a perspective view of a surgical tool according to another embodiment of the present invention wherein a universal joint is provided between the holder and the weight; and

FIG. 8 is a perspective view of the universal joint illustrated in FIG. 7.

### DETAILED DESCRIPTION OF THE PREFERRED EMBODIMENTS

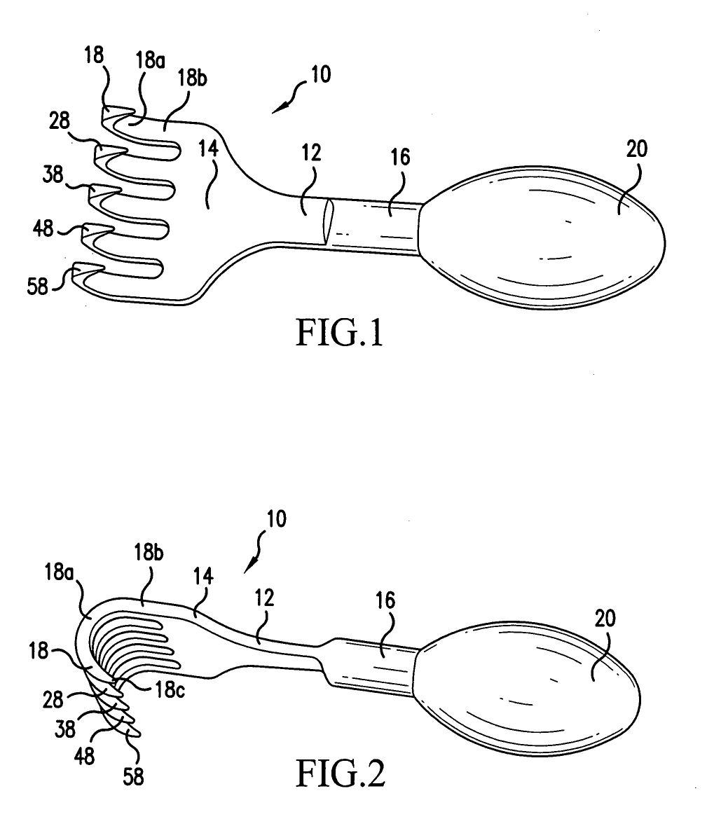

As illustrated in FIGS. 1 and 2 a surgical instrument 10 according to a first embodiment of the present invention is provided for maintaining an incision in an opened condition during an operation. A holder 12 includes a distal end 14 and a proximal end 16. At least one projection 18 is provided at the distal end 14 of the holder 12 wherein the at least one projection 18 is adapted to engage a peripheral edge of an incision for maintaining the incision in an open condition. A predetermined weight 20 is formed on the proximal end 16 of the holder 12 for providing a predetermined gravitational force for maintaining the holder 12 in a predetermined position for maintaining the incision in an open condition.

As illustrated in FIGS. 1 and 2, the at least one projection 18 initially extends forwardly away from the distal end 14 and is curved 18a to extend downwardly and rearwardly towards the proximal end 16.

The at least one projection 18 may include a plurality of additional projections 28, 38, 48 and 58 that are adapted for engaging a plurality of locations along a peripheral edge of an incision for maintaining an elongated incision in an open condition. It is anticipated that the surgical tool 10 will be used on both sides along a peripheral edge of an incision for maintaining the incision in an open condition. However, depending on the surgery the surgical tool 10 may be positioned along one side of the incision. In addition, depending on the operation and the length of the incision, more than one surgical tool 10 may be used along the length of the incision to maintain the incision in an open condition.

As illustrate in FIGS. 1 and 2, the least one projection 18 includes a flat portion 18b extending from the distal end 14 of the holder 12 a predetermined distance and includes a pointed end 18c adapted to engage a peripheral edge of an incision for maintaining the incision in an open condition. If a plurality of projections 18, 28, 38, 48 and 58 are provided, each of the plurality of projections 18, 28, 38, 48 and 58 includes a flat portion extending from the distal end 14 of the holder 12 a predetermined distance and a pointed end adapted to engage a peripheral edge of an incision with each of the plurality of projections being adapted for engaging a plurality of locations along a peripheral edge of an incision for maintaining the incision in an open condition.

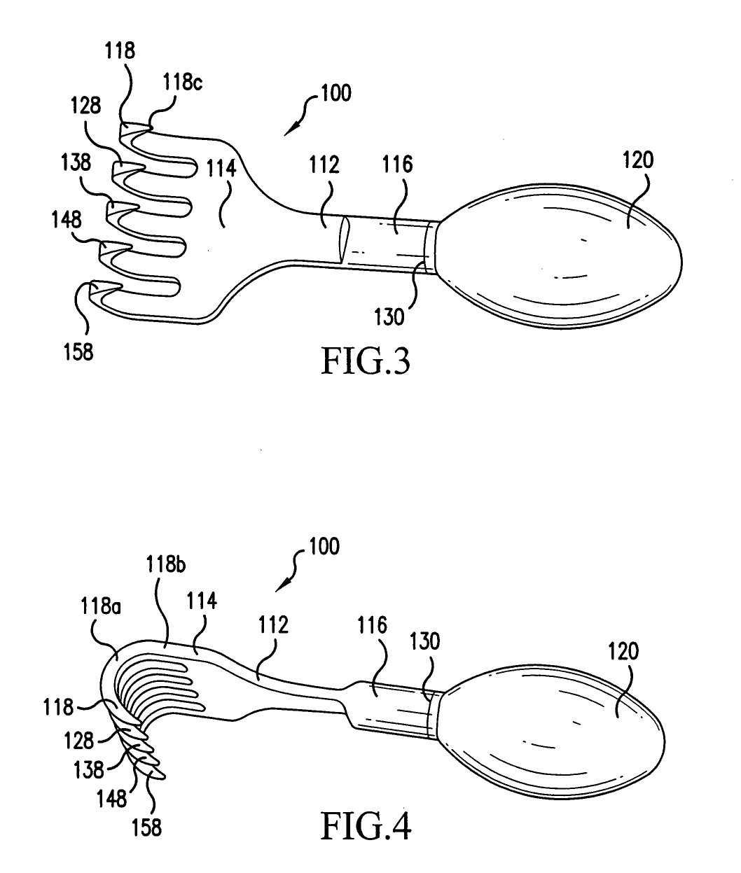

As illustrated in FIGS. 3-6, a surgical instrument 100 according to a second embodiment of the present invention is also provided for maintaining an incision in an opened condition during an operation. A holder 112 includes a distal end 114 and a proximal end 116. At least one projection 118 is provided at the distal end 114 of the holder 112 wherein the at least one projection 118 is adapted to engage a peripheral edge of an incision for maintaining the incision in an open condition. A predetermined weight 120 is formed on the proximal end 116 of the holder 112 for providing a predetermined gravitational force for maintaining the holder 112 in a predetermined position for maintaining the incision in an open condition.

As illustrated in FIGS. 3-6, the surgical instrument 100 according to the second embodiment of the present invention includes an attachment mechanism 130 formed on the proximal end 116 of the holder 112 for selectively attaching one of a plurality of weights 120, 220, 320, 420, 520 or 620 to the proximal end 116 for obtaining the predetermined gravitational force depending on the incision.

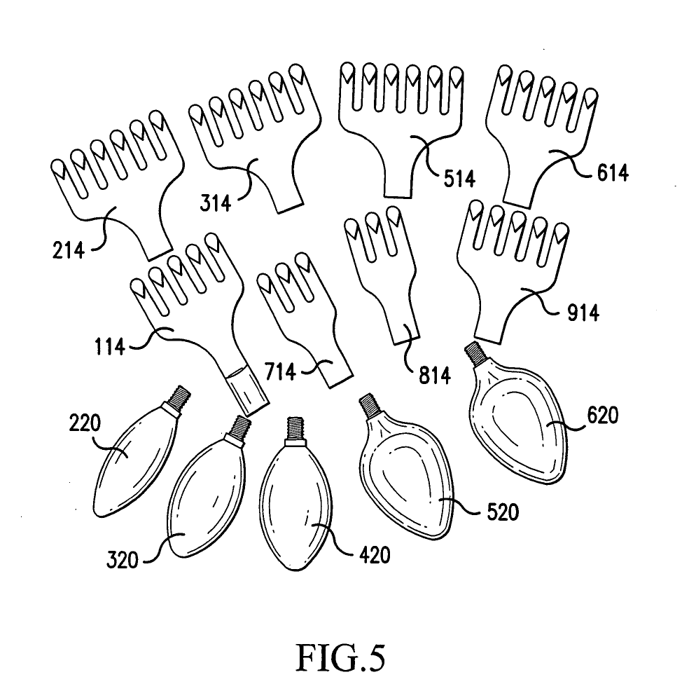

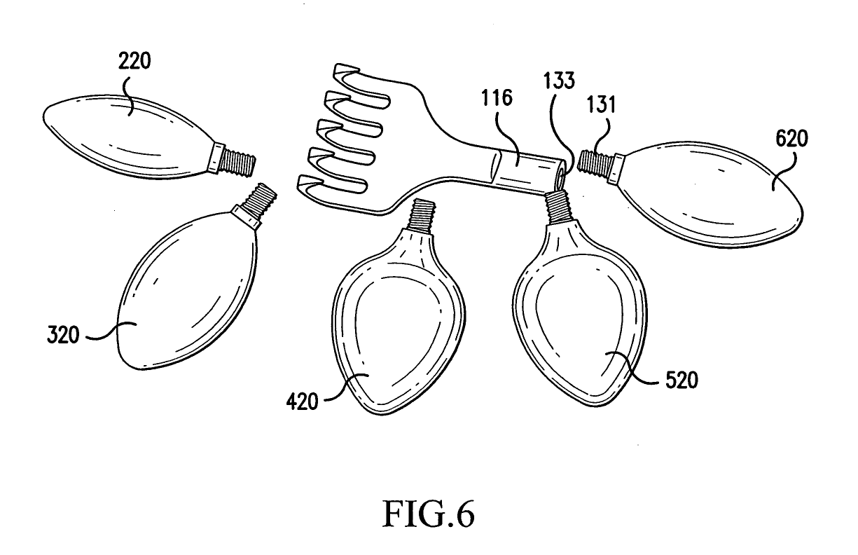

As illustrated in FIGS. 5 and 6, the surgical instrument 100 may include an attachment mechanism 130 that is a male screw thread 131 formed on each of the weights and a female screw threaded opening 133 formed in the proximal end 116 for selectively attaching one of a plurality of weights 120, 220, 320, 420, 520 or 620 to the proximal end 116 of the holder 112. It is understood that the male and female screw threads may be reversed wherein the proximal end 116 may include the male screw thread 131 and the female screw thread 133 may be formed on the weights. In addition, other forms of an attachment mechanism 130 are envisioned in the present invention for removably securing the weights 120, 220, 320, 420, 520 or 620 to the proximal end 116 of the holder 112.

As illustrated in FIGS. 3 and 4, the at least one projection 118 initially extends forwardly away from the distal end 114 and is curved 118a to extend downwardly and rearwardly towards the proximal end 116.

The at least one projection 118 may include a plurality of additional projections 128, 138, 148 and 158 that are adapted for engaging a plurality of locations along a peripheral edge of an incision for maintaining an elongated incision in an open condition.

As illustrate in FIGS. 3 and 4, the least one projection 118 includes a flat portion 118b extending from the distal end 114 of said holder 112 a predetermined distance and includes a pointed end 118c adapted to engage a peripheral edge of an incision for maintaining the incision in an open condition. If a plurality of projections 118, 128, 138, 148 and 158 are provided, each of the plurality of projections 118, 128, 138, 148 and 158 includes a flat portion extending from the distal end 114 of the holder 112 a predetermined distance and a pointed end adapted to engage a peripheral edge of an incision with each of the plurality of projections being adapted for engaging a plurality of locations along a peripheral edge of an incision for maintaining the incision in an open condition.

The surgical instrument 10, 100 provides a space between the distal end 14, 114 of the holder 12, 112 and the at least one projection 18, 118 with the distal end 14, 114 and the holder 12, 112 being spaced a predetermined distance relative to each other for accommodating a thickness of a peripheral edge of an incision.

The surgical instrument 10, 100 provides at least one projection 18, 118 that may be formed as a plurality of projections arranged side by side in a tines of a fork like arrangement with a pointed end 18c, 118c being adapted to engage a peripheral edge of an incision projecting towards the proximal end 16, 116 thereof.

The surgical instrument 100 provides the distal end 114 that includes a flat portion forming the at least one projection 118 and an attachment portion 130 for selectively being attached to one of a plurality of weights 120, 220, 320, 420, 520 or 620. The attachment portion 130 including an enlarged section for mating with an enlarged section of one of the plurality of weights for attachment thereto. The attachment portion 130 may be a friction fit for securely maintaining the proximal end 116 to one of the plurality of weights 120, 220, 320, 420, 520 or 620.

As illustrated in FIG. 5, the holder 112 may include a plurality of various distal end members 114, 214, 314, 414, 514, 614, 714, 814 or 914 that may be selectively attached to one of a plurality of weights 220, 320, 420, 520 or 620 depending on the incision that is to be maintained in an open condition.

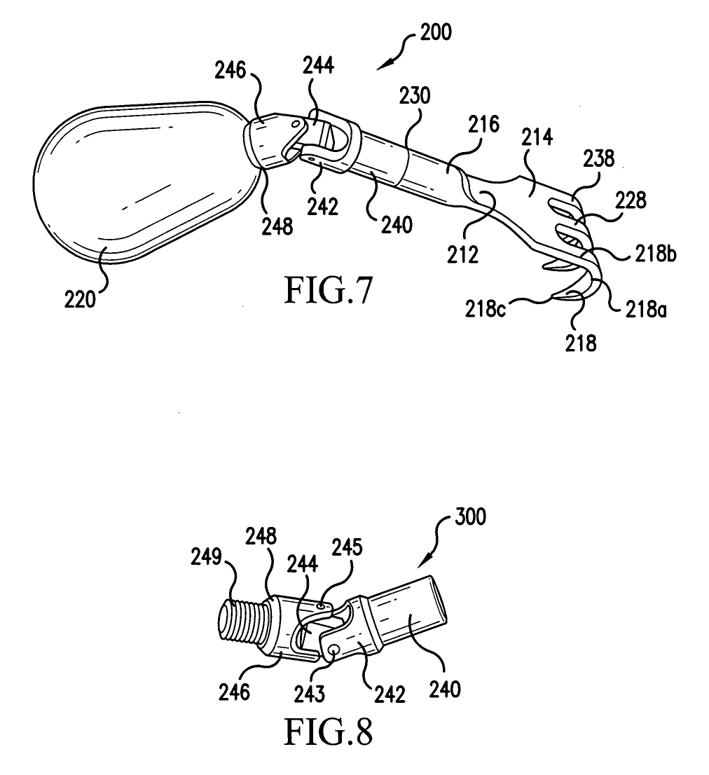

As illustrated in FIGS. 7 and 8, a surgical instrument 200 is provided according to another embodiment of the present invention. The surgical instrument 200 includes a holder 212 with a distal end 214 and a proximal end 216. At least one projection 218 is provided at the distal end 214 of the holder 212 wherein the at least one projection 218 is adapted to engage a peripheral edge of an incision for maintaining the incision in an open condition. A predetermined weight 220 is mounted to one end of a universal joint 300. The universal joint 300 provides correct positioning of the holder 212 relative to the weight 220 to permit a surgeon to move the holder 212 relative to the weight 220. The universal joint 300 includes a first end 240 that is operatively connected to the proximal end 216 of the holder 212 at the joint 230. A cage or second end 242 is connected to the first end 240 and is hinged by a pivot 243 to a connector 244. The universal joint 300 includes a second end 246 that is operatively connected to the weight 220 at a joint 248. The second end 246 may include a male threaded section 249 for attachment to a female threaded section of the weight 220. In the alternative, the male threaded portion may be formed on the weight 220 and the second end 246 may include a female threaded section for mating with the male threaded section on the weight 220. The second end 246 includes a cage connected to the second end 246 and is hinged by a pivot 245 to the connector 244.

As illustrated in FIGS. 7 and 8, the surgical instrument 200 may include an joint 230 that is a female screw thread for attachment of the first end 240 of the universal joint 300 to the proximal end 216 of the holder 212. It is understood that the male and female screw threads may be reversed wherein the first end 240 may include the male screw thread and the female screw thread may be formed on the proximal end 216 of the holder 212. In addition, other forms of joint 230 are envisioned in the present invention for attaching the universal joint 300 to the proximal end 216 of the holder 112.

As illustrated in FIG. 7, the at least one projection 218 initially extends forwardly away from the distal end 214 and is curved 218a to extend downwardly and rearwardly towards the proximal end 116.

The at least one projection 218 may include a plurality of additional projections 228 and 238 that are adapted for engaging a plurality of locations along a peripheral edge of an incision for maintaining an elongated incision in an open condition.

As illustrate in FIG. 7, the least one projection 218 includes a flat portion 218b extending from the distal end 214 of said holder 212 a predetermined distance and includes a pointed end 218c adapted to engage a peripheral edge of an incision for maintaining the incision in an open condition. If a plurality of projections 218, 228 and 238 are provided, each of the plurality of projections 218, 228 and 238 includes a flat portion extending from the distal end 214 of the holder 212 a predetermined distance and a pointed end adapted to engage a peripheral edge of an incision with each of the plurality of projections being adapted for engaging a plurality of locations along a peripheral edge of an incision for maintaining the incision in an open condition.

The invention being thus described, it will be obvious that the same may be varied in many ways. Such variations are not to be regarded as a departure from the spirit and scope of the invention, and all such modifications as would be obvious to one skilled in the art are intended to be included within the scope of the following claims.

What is claimed is:

1. A surgical instrument for maintaining an incision in an opened condition during an operation comprising:
a holder having a distal end and a proximal end;
at least one projection being provided at the distal end of said holder, said at least one projection being adapted to engage a peripheral edge of an incision for maintaining the incision in an open condition; and
a predetermined weight being formed on the proximal end of said holder for providing a predetermined gravitational force for maintaining said holder in a predetermined position for maintaining the incision in an open condition.

2. The surgical instrument according to claim 1, wherein said at least one projection initially extends forwardly away from the distal end and is curved to extend downwardly and rearwardly towards said proximal end.

3. The surgical instrument according to claim 2, wherein said at least one projection includes a plurality of projections adapted for engaging a plurality of locations along a peripheral edge of an incision for maintaining an elongated incision in an open condition.

4. The surgical instrument according to claim 2, wherein said at least one projection includes a flat portion extending from the distal end of said holder a predetermined distance and including a pointed end adapted to engage a peripheral edge of an incision for maintaining the incision in an open condition.

5. The surgical instrument according to claim 1, wherein said at least one projection includes a plurality of projections, each of said plurality of projections including a flat portion extending from the distal end of said holder a predetermined distance and a pointed end adapted to engage a peripheral edge of an incision, wherein each of said plurality of projections is adapted for engaging a plurality of locations along a peripheral edge of an incision for maintaining the incision in an open condition.

6. The surgical instrument according to claim 1, wherein said proximal end of said holder includes an attachment mechanism for selectively attaching one of a plurality of weights for obtaining the predetermined gravitational force depending on the incision.

7. The surgical instrument according to claim 6, wherein said attachment mechanism is a screw thread wherein at least one of said proximal end and said weights includes one of a male and female thread for selectively attaching one of a plurality of weights to the proximal end of said holder.

8. The surgical instrument according to claim 1, wherein the distal end of the holder and the at least one projection are spaced a predetermined distance relative to each other for accommodating a thickness of a peripheral edge of an incision.

9. The surgical instrument according to claim 1, wherein said at least one projection is a plurality of projections arranged side by side in a tines of a fork like arrangement with a pointed end adapted to engage a peripheral edge of an incision projecting towards the proximal end thereof.

10. The surgical instrument according to claim 1, wherein said distal end includes a flat portion forming the at least one projection and an attachment portion for selectively being attached to one of a plurality of weights, said attachment portion including an enlarged section for mating with an enlarged section of one of the plurality of weights for attachment thereto.

11. The surgical instrument according to claim 1, and further including a universal joint for operatively connecting said proximal end of said holder to the predetermined weight for positioning the holder relative to the predetermined weight.

12. A surgical instrument for use in maintaining an incision in an opened condition comprising:
a holder having a distal end and a proximal end;
at least one projection being formed at the distal end of said holder, said at least one projection being adapted to engage a peripheral edge of an incision for maintaining the incision in an open condition; and
said proximal end of said holder providing a predetermined gravitational force for maintaining said holder in a predetermined position for maintaining the incision in an open condition.

13. The surgical instrument according to claim 12, wherein said at least one projection initially extends forwardly away from the distal end and is curved to extend downwardly and rearwardly towards said proximal end.

14. The surgical instrument according to claim 13, wherein said at least one projection includes a plurality of projections adapted for engaging a plurality of locations along a peripheral edge of an incision for maintaining an elongated incision in an open condition.

15. The surgical instrument according to claim 13, wherein said at least one projection includes a flat portion extending from the distal end of said holder a predetermined distance and including a pointed end adapted to engage a peripheral edge of an incision for maintaining the incision in an open condition.

16. The surgical instrument according to claim 12, wherein said at least one projection includes a plurality of projections, each of said plurality of projections including a flat portion extending from the distal end of said holder a predetermined distance and a pointed end adapted to engage a peripheral edge of an incision, wherein each of said plurality of projections is adapted for engaging a plurality of locations along a peripheral edge of an incision for maintaining the incision in an open condition.

17. The surgical instrument according to claim 12, wherein said proximal end of said holder includes an attachment mechanism for selectively attaching one of a plurality of weights for obtaining the predetermined gravitational force depending on the incision.

18. The surgical instrument according to claim 17, wherein said attachment mechanism is a screw thread wherein at least one of said proximal end and said weights includes one of a male and female thread for selectively attaching one of a plurality of weights to the proximal end of said holder.

19. The surgical instrument according to claim 12, wherein the distal end of the holder and the at least one projection are spaced a predetermined distance relative to each other for accommodating a thickness of a peripheral edge of an incision.

20. The surgical instrument according to claim 12, wherein said at least one projection is a plurality of projections arranged side by side in a tines of a fork like arrangement with a pointed end adapted to engage a peripheral edge of an incision projecting towards the proximal end thereof.

21. The surgical instrument according to claim 12, wherein said distal end includes a flat portion forming the at least one projection and an attachment portion for selectively being attached to one of a plurality of weights, said attachment portion including an enlarged section for mating with an enlarged section of one of the plurality of weights for attachment thereto.

22. The surgical instrument according to claim 12, and further including a universal joint for operatively connecting said proximal end of said holder to the predetermined weight for positioning the holder relative to the predetermined weight.
